(19) United States
(12) Patent Application Publication
(10) Pub. No.: US 2021/0070730 A1
(43) Pub. Date: Mar. 11, 2021

(54) H2S-BASED THERAPEUTIC AGENTS FOR THE TREATMENT OF NEURODEGENERATIVE DISEASES

(71) Applicant: INTERNATIONAL SOCIETY FOR DRUG DEVELOPMENT S.R.L., MILANO (IT)

(72) Inventors: Simona Rapposelli, LUCCA (IT); Simona Sestito, CHIARAVALLE CENTRALE (IT); Vincenzo Calderone, MASSA (IT); Guido Puricelli, MILANO (IT)

(73) Assignee: INTERNATIONAL SOCIETY FOR DRUG DEVELOPMENT S.R.L., MILANO (IT)

(21) Appl. No.: 16/957,482

(22) PCT Filed: Oct. 8, 2018
(86) PCT No.: PCT/EP2018/077343

§ 371 Date: Jun. 24, 2020

(30) Foreign Application Priority Data
Dec. 27, 2017 (IT) 102017000149921

(51) Int. Cl. C07D 339/04; C07C 331/26; A61K 31/26; A61K 31/385; A61K 31/13

(52) U.S. Cl. CPC C07D 339/04; C07C 331/26; C07C 2603/74; A61K 31/385; A61K 31/13; A61K 31/26

(57) ABSTRACT

The present invention concerns the field of neurodegenerative diseases, and in particular relates to compounds, pharmaceutical compositions and their uses in the protection of neuronal cells from inflammation and from oxidative stress in the early stage of Parkinson's Disease as well as in Alzheimer's Disease.

### FIELD OF THE INVENTION

The present invention concerns the field of neurodegenerative diseases, and in particular relates to compounds, pharmaceutical compositions and their uses in the protection of neuronal cells from inflammation and from oxidative stress in the early stages of Parkinson's Disease as well as in Alzheimer's Disease.

### STATE OF THE ART

Hydrogen sulfide (H₂S) is emerging as a hot topic in the field of drug discovery. H₂S is a well-known pungent and toxic gas and has been recognized as the third gaseous signaling molecule in addition to nitric oxide and carbon monoxide [1]. H₂S is produced endogenously from the amino acids L-cysteine and homocysteine by several enzymes. In particular, in the brain it is synthesized by cystathionine-b-synthetase (CBS) which is highly expressed in the hippocampus and cerebellum [2]. Noteworthy, a dramatic decrease of CBS activity and a consequent drastic fall in H₂S levels (about 50%) have been detected in the brain of patients affected by Alzheimer's disease (AD). Moreover, it seems to play a neuroprotective role in Parkinson's disease [3], thus suggesting that this gaseous-transmitter is able to prevent or halt the pathological state in several neurodegenerative diseases. A rapid increase in the knowledge on the biological functions of H₂S prompted to deeply investigate the pharmacological effects of H₂S as neuromodulator, neuroprotectant and anti-inflammatory agent. In particular, H₂S has been recognized as neuromodulator [2, 4] via the involvement of at least two classes of ionotropic glutamate receptors, which play critical roles in synaptic plasticity, NMDA and AMPA receptors [4, 5]. Even if the mechanism of action needs to be further elucidated, H₂S acts both directly on NMDA receptor (via sulphydrating cysteine residues) [6] and indirectly, through the regulation of intracellular Ca⁺² levels [7]. Furthermore, both in vitro and in vivo experiments showed that H₂S plays a neuroprotective role in AD and PD. Indeed, NaHS (a well-known inorganic salt and precursor of H₂S) induces neuroprotection against oxidative stress through at least three main mechanisms: (a) restoration of cellular levels of GSH (glutathione) [8]; (b) activation of ATP sensitive potassium channels(KATP); (c) decreasing mitochondrial ROS production [9, 10]. Consistently with the critical role in neuroprotection, H₂S plays a crucial role also in neuroinflammation. NaHS proved to dramatically reduce the release of proinflammatory cytokines such as TNF-α, IL-1β and IL-6 induced by amiloyd β-peptides. Moreover, it inhibits the upregulation of COX₂ enzyme and the activation of NF-kB in the hippocampus [11], thus reiterating the high potential value of H₂S in AD therapy. Recent evidences showed also that inhaled H₂S in a mouse model of Parkinson's disease induced by MPTP was associated with upregulation of genes encoding antioxidant proteins [3], including hemeoxygenase-1 and glutamate-cysteine ligase. Altogether, these observations suggest that H₂S could prevent neurodegeneration also via an upregulation of antioxidant defense mechanisms and inhibition of inflammation and apoptosis in the brain. Moreover, many other mechanisms have been hypothesized. Among them, H₂S seems to protect neurons against oxidative stress via activation of upstream receptor tyrosine kinase. Recent findings reported that H2S inhibits lipopolysaccharide (LPS)-induced nitric oxide (NO) production in microglia via inhibition of p38-MAPK, a signaling pathway that regulates cellular activities, such as apoptosis, differentiation, metabolism. As a whole, these findings corroborate the functional involvement of H₂S in neurodegenerative diseases [12, 13]. Therefore, the restoration of the correct levels of endogenous H₂S is an appealing challenge for the development of new potential therapies for neurodegenerative diseases.

Recently, it has been also proven that H₂S has protective effects against A-beta-induced cell injury by inhibiting inflammation, promoting cell growth, and preserving mitochondrial function [2, 14]. Given that the main drawback of AD therapy still remains the limited effectiveness, the search for new potential drugs is heavily pursued. In the last two decades, the multitarget-directed-ligand (MTDL) strategy has raised considerable attention. The development of a single molecule with synergistic actions has been successfully realized for the treatment of several kinds of multi-factorial diseases such as cancer and cardiovascular disease. Since the pathological state of AD is the result of a network impairment, many multifunctional agents have been synthesized for the treatment of memory and cognition impairments which interact simultaneously with two or more targets such as AChE, β-amyloid (Aβ), tau protein, monoamine oxidase, metal ions, reactive oxygen species (ROS) and many others.

Based on current knowledge about the pathophysiological actions of endogenous H₂S in CNS, the pharmacological modulation of such an important gaseous mediator is becoming a challenging field of research in drug discovery. The administration of gaseous H₂S is greatly limited by the difficulty to ensure an accurate dosage and the risk of overdose (with dramatic consequences due to H₂S toxicity). For these reasons, the use of H₂S-releasing compounds seems to be the most convenient and compelling strategy [15-17].

The need and importance is increasingly felt for the development of novel multitarget molecules able to protect neuronal cells from inflammation and oxidative stress in the early stage of PD as well as in AD.

It is therefore object of the present invention the development and the design of compounds which allow to restore H₂S level in the CNS, and to affect the alteration in the pathways involved in neuroinflammation processes and in mitochondria dysfunctions, thus delaying the neurodegeneration process and, consequently, the disease progression.

### SUMMARY OF THE INVENTION

The present invention concerns a compound of formula (I)

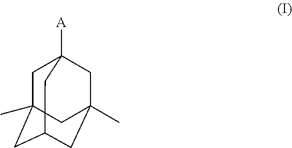

or a pharmaceutical salt thereof

wherein:

A is —N═C═S or —NH—B

where B is

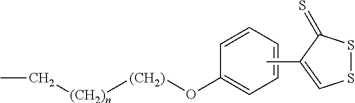

and n=0-3

for use as in the treatment of a neurodegenerative disease.

The problem underlying the present invention is that of making available compounds capable of restoring H₂S levels in the CNS, in order to permit the manufacture of medicaments destined to delaying the neurodegeneration process and consequently, neurodegenerative disease progression.

This problem is solved by the present finding by the use of a compound capable of restoring the correct levels of endogenous H₂S for the treatment of neurodegenerative diseases such as Alzheimer's disease and Parkinson's disease.

The invention further provides for a method for treating or preventing the development of a neurodegenerative disease in a subject in need thereof, said method comprising administering a therapeutically effective amount of a compound or a pharmaceutical composition according to the present invention to the subject, thereby treating or reducing the risk of developing a neurodegenerative disease.

In a further aspect, the invention concerns a compound of formula (I)

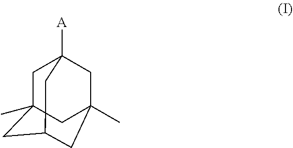

or a pharmaceutical salt thereof

wherein:

A is —NH—B,

where B is

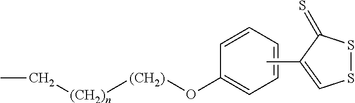

and n=0-3.

In a further embodiment the invention provides a pharmaceutical composition comprising a compound of formula (I) of the invention, and one or more pharmaceutically acceptable excipients.

As will be further described in the detailed description of the invention, the compounds described herein further allow the development of pharmaceutical products which may be used in combination with drugs already used in the treatment of cognitive decline, allowing an improved neuroprotective pharmacological profile.

### BRIEF DESCRIPTION OF THE DRAWINGS

The characteristics and advantages of the present invention will be apparent from the detailed description reported below, from the Examples given for illustrative and non-limiting purposes, and from the annexed FIGS. 1-7, wherein:

FIG. 1: Curves describe the increase of H₂S concentration, with respect to time, following the incubation of GYY 4137, the H₂S-prodrug MT66 in the assay buffer, in the absence (white symbol) or in the presence of L-cysteine (black symbol) or glutathione (black triangles). H₂S was recorded by amperometry; the vertical bars indicate the SEM.

FIG. 2: Neuroprotective effects. Human neuronal-like cells were treated with medium alone (CONTROL) or with the compounds (10 μM) for 24 h (light grey) or 72 h(dark grey); after drug removal, cells were incubated with 50 μg/ml LPS and 50 ng/ml TNF-α for an additional 16 h. At the end of treatment, cell proliferation was measured by MTS assay. The data are expressed as percentages relative to untreated cells (control), which were set at 100%, and represent the mean±SEM of three independent experiments, each performed in triplicate. Statistical significance was determined using a one-way ANOVA-Tukey HSD post hoc test: *P<0.05, ***P<0.001 vs. control; ##P<0.01, ###P<0.001 vs. cells treated with LPS-TNF-α.

FIG. 3. Effects induced on ROS production. Human neuronal-like cells were treated with medium alone (CONTROL) or the compound (10 μM) for 24 h; after drug removal, cells were incubated with 50 μg/ml LPS and 50 ng/ml TNF-α for an additional 16 h. At the end of treatment, ROS production was measured using the fluorogenic probe DCFH₂-DA. The data are expressed as percentages relative to untreated cells (control), which were set at 100%, and represent the mean±SEM of two independent experiments, each performed in triplicate. Statistical significance was determined using a one-way ANOVA-Tukey HSD post hoc test: ***P<0.001 vs. control; ###P<0.001 vs. cells treated with LPS-TNF-α.

FIG. 4. Effects of the novel compounds on Abeta aggregation.

FIG. 5. Effect of the compounds on HepG2 cells. The data are expressed as a percentage with respect to that of vehicle treated cells (DMSO) which was set to 100% (mean values±SEM, N=3). *p<0.05 vs vehicle treated cells; **p<0.01 vs Memantine 1 μM treated cells; #p<0.05 vs Memantine 10 μM treated cells.

FIG. 6. Western blot quantification of LC3II/LC3I ratio, p62, mTOR and Akt as hallmarks of the degree of ATG activation. After 4 hours, the compounds (10 μM) and Rapamycin (1 μM) induced (A) increased LC3II/LC3I ratio, (B) a significant p62 degradation and (C) decreased p-mTOR/mTOR ratio in U87MG cells. (D) Western blot quantification of pAkt/Akt ratio in U87MG cells. Results represent mean±SEM of three different gels. *P<0.05, **P<0.01, ***P<0.001 versus vehicle treated cells (Control).

FIG. 7. Rat microglia cells were pre-treated with the compounds (10 μM) for 24 h. After washing, the cells were incubated with Aβ1-42 for 24 h. At the end of treatments, cell proliferation was measured by MTS assay. The data are expressed as percentages relative to untreated cells (control), which were set at 100%, and represent the mean±SEM of three independent experiments, each performed in triplicate. Statistical significance was determined using a one-way ANOVA followed by a Bonferroni post-test: *p<0.05 vs control; #p<0.05 vs cells treated with Aβ1-42.

### DETAILED DESCRIPTION OF THE INVENTION

In a first aspect, the present invention concerns a compound of formula (I)

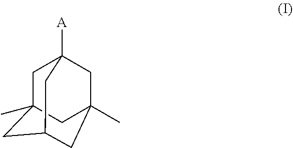

wherein:

A is —N═C═S or —NH—B

where B is

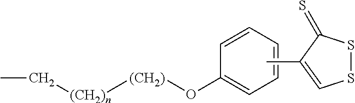

and n=0-3 for use in the treatment of a neurodegenerative disease.

The compounds according to the present invention are active molecules which have surprisingly been seen involved in the release of H₂S and thus capable of delaying neurodegenerative disease progression.

The compounds according to the present invention have been identified and synthesized and have been demonstrated as being multitarget molecules that are able to protect neuronal cells from inflammation and oxidative stress in the early stage of PD as well as in AD.

In particular, exploiting their ability to restore H₂S level in CNS, the new derivatives have surprisingly been seen to affect the alteration in the pathways involved in the neuroinflammation process and in mitochondria dysfunction, thus delaying the neurodegeneration process and, consequently, the disease progression.

Preferably in the first aspect, A is —N═C═S.

When A is —NH—B, in the group B n is preferably 1.

When in the present invention, it is referred to a neurodegenerative disease, it is intended a disease selected from the group of chronic, progressive disorders characterized by the gradual loss of neurons in discrete areas of the central nervous system (CNS).

Preferably, the neurodegenerative disease is a disease selected from the group consisting of Parkinson's disease, Alzheimer's disease, amyotrophic lateral sclerosis and Huntington's disease.

The mechanism(s) underlying the progressive nature of such a neurodegenerative disease remains unknown but a timely and well-controlled inflammatory reaction is essential for the integrity and proper function of the CNS.

In a further aspect, the invention concerns a compound of formula (I)

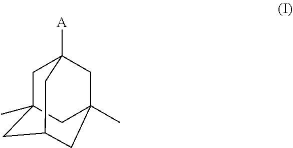

or a pharmaceutical salt thereof

wherein:

A is —NH—B,

where B is

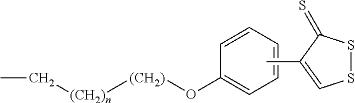

and n=0-3.

Preferably, n is 1.

In a further embodiment the invention provides a pharmaceutical composition comprising a compound of formula (I) of the invention, and at least one pharmaceutically acceptable excipient.

The pharmaceutical composition according to the present invention is preferably for intravenous, oral, intrathecal, intranasal, intraperitoneal or intramuscular administration.

The pharmaceutical compositions according to the present invention can be used alone or in combination with one or more further drugs.

In particular, Memantine of Formula,

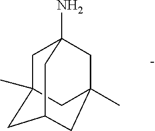

as NMDA antagonist (N-Methyl-D-aspartate receptor antagonist), is a drug currently used for the treatment of cognitive decline in PD and AD. In another aspect the inventors propose to combine Memantine with at least one compound or the pharmaceutical composition according to the present invention, in order to obtain new chemical entities with a better neuroprotective pharmacological profile than the “native drug”.

Therefore, the invention concerns also a pharmaceutical composition comprising a compound of Formula (I), wherein A is —N═C═S or —NH—B and Memantine as active ingredients.

The invention further provides for a method for treating or preventing the development of a neurodegenerative disease in a subject in need thereof, said method comprising administering a therapeutically effective amount of a compound or a pharmaceutical composition according to the present invention to the subject, thereby treating or reducing the risk of developing a neurodegenerative disease.

In a preferred aspect, in the method for treating or preventing the development of a neurodegenerative disease in a subject, said neurodegenerative disease is chosen from the group consisting of Alzheimer's disease, Parkinson's disease amyotrophic lateral sclerosis and Huntington's disease.

Reference is now made to the following examples, which together with the above descriptions illustrate some embodiments of the invention.

### EXAMPLES

### Example 1

### Preparation of the Compounds of Formula (I) When A is —N═C═S

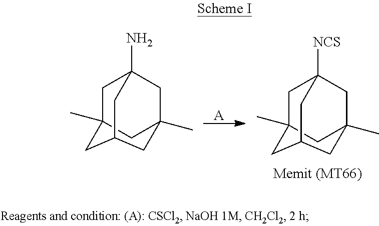

Synthetic Procedure to Synthesize Compound Memit (MT66).

To a stirred solution of memantine (300 mg, 1.67 mmol) in CH₂Cl₂ (15 ml) and NaHCO₃ 6% (15 ml) cooled at 0° C., was added dropwise thiophosgene (1.28 ml, 16.7 mmol). The resulting mixture was stirred at 2 h at r.t, followed by TLC, then the aqueous phase was separated and extracted several times with CH₂Cl₂, dried over anhydrous Na₂SO₄ and concentrated. The crude product MT66 was purified through flash chromatography (AcOEt/n-hexane 9:1 as the eluent) to get the final product as a clear oil. ¹H NMR (CDCl₃ −400 MHz): δ 0.84 (s, 6H), 1.12 (s, 2H), 1.24-1.34 (s, 4H), 1.47-1.69 (m, 4H), 1.77-1.78 (m, 2H), 2.11-2.16 (m, 1H) ppm.

### Example 2

### Preparation of the Compounds of Formula (I) When A is —NHB, Wherein in B n is 1

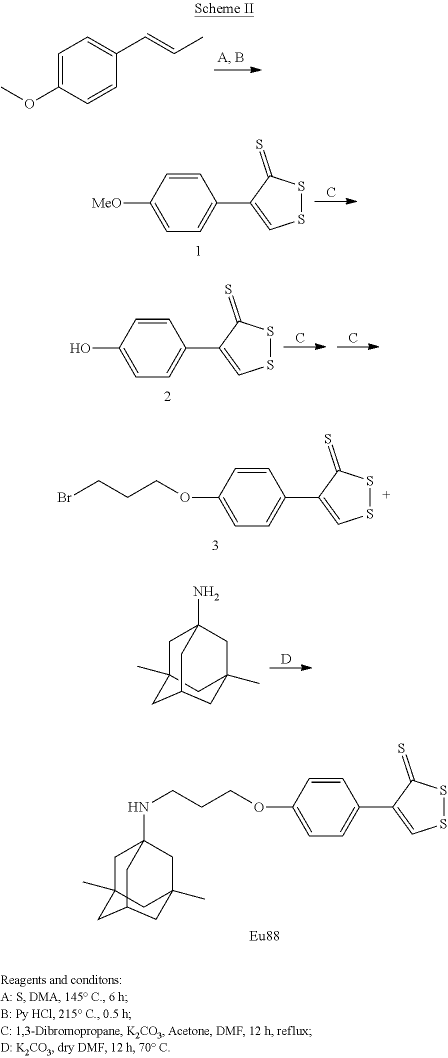

4-(4-methoxyphenyl)-3H-1,2-dithiole-3-thione(1): Sulfur (1.5 g, 6.75 mmol), trans-anethole (1 g, 6.75 mmol), e DMA (3.37 ml) were heated at 145° C. for 6 h. Then the mixture was concentrated, rinsed with Et₂O and the resulting solid was filtered off. The resulting organic solution was concentrated and purified through flash chromatography using n-hexane/AcOEt 9:1 as the eluent, affording the intermediate 1 as a pure product. ¹H NMR (CDCl₃ −400 MHz): δ 3.83 (s, 3H, OCH3), 6.68 (d, 2H, J=8.8 MHz), 7.40 (s, 1H), 7.62 (d, 2H, J=8.8 MHz) ppm.

4-(4-hydroxyphenyl)-3H-1, 2-dithiole-3-thione(2): 4-(4-methoxyphenyl)-3H-1,2-dithiole-3-thione (570 mg, 2.37 mmol) and pyridine hydrochloride (2.85 g) were heated at 215° C. for 25 min. Then the reaction was cooled to r.t. and diluted with HCl 1N. The resulting precipitate was filtered, washed HCl 1N and dried under reduced pressure to yield 4-(4-hydroxyphenyl)-3H-1,2-dithiole-3-thione, used for the following step without further purifications. ¹H NMR (MeOH-d₄ −400 MHz): δ 6.89 (d, 2H, J=8.8 MHz), 7.48 (s, 1H), 7.67 (d, 2H, J=8.8 MHz) ppm.

4-(4-(3-bromopropoxy)phenyl)-3H-1,2-dithiole-3-thione (3): To a solution of 4-(4-hydroxyphenyl)-3H-1,2-dithiole-3-thione (2) (208 mg, 0.92 mmol) in acetone was added K₂CO₃ (636 mg, 4.6 mmol) and 1,3-dibromopropane (557 mg, 2.76 mmol). The reaction mixture was refluxed overnight, then cooled and filtered. The organic solution obtained was concentrated and purified by flash chromatography using as eluent a mixture of Petroleum ether/Acetone (9:1). ¹H NMR (CDCl₃ −400 MHz): δ 2.33-2.39 (m, 2H), 3.61 (t, 2H, J=6.0 MHz), 4.18 (t, 2H, J=6.0 MHz), 6.98 (d, 2H, J=8.8 MHz), 7.40 (s, 1H), 7.62 (d, 2H, J=8.8 MHz) ppm.

4-(4-(3,5-dimethyladamantan-1-yl)amino)propoxy)phenyl)-3H-1,2-dithiole-3-thione (Eu88): 4-(4-(3-bromopropoxy)phenyl)-3H-1,2-dithiole-3-thione (3) (100 mg, 0.28 mmol) and K₂CO₃ (119 mg, 0.86 mmol) were stirred in dry DMF under N₂ atmosphere. Memantine hydrochloride (75 mg, 0.35 mmol) was added and the resulting mixture heated overnight at 70° C. Then the mixture was concentrated under vacuum and rinsed with CH₂Cl₂. The suspension was filtered and the organic phase dried over anhydrous Na₂SO₄ and concentrated. The crude product was purified by flash chromatography over silica gel, using 0-10% MeOH as a gradient in CHCl₃. Derivative Eu88 so obtained was then transformed into the corresponding chloride salt. ¹H NMR (DMSO-d6 −400 MHz): δ 0.87 (s, 6H), 1.10-1.19 (m, 2H), 1.29-1.32 (m, 4H), 1.50-1.57 (m, 4H), 1.73-1.74 (m, 2H), 2.10-2.17 (m, 2H), 2.17-2.19 (m, 1H), 3.01-3.04 (m, 2H), 4.19 (t, 2H, J=6.0 MHz), 7.10 (d, 2H, J=8.8 MHz), 7.78 (s, 1H), 7.90 (d, 2H, J=8.8 MHz), 8.87-8.9 (br s, 2H, NH₂⁺) ppm. ¹³C NMR (DMSO-d6 −400 MHz): δ 214.83, 173.69, 161.71, 134.22, 129.01, 123.84, 115.54, 65.32, 57.75, 49.41, 43.45, 41.47, 36.42, 36.25, 32.07, 29.61, 29.11, 25.95 ppm.

### Example 3

### In Vitro Evaluation of the Pharmacodynamics (PD) and Pharmacokinetics (PK) of the Compounds of the Invention

Although the pharmacological properties of the “native” drugs Memantine have been extensively studied, the conjunction with a H₂S-donor moiety creates a new molecule, such as MT66 with different/additional properties when compared to the native drug (i.e. NMDA antagonism and H₂S releasing properties) that needs to be investigated.

Therefore, the compound MT66 has been investigated for its neuroprotective activity in human neuroblastoma cell lines (SH-SY5Y, H9 neuronal stem cells) or primary rat hippocampal neurons culture (i.e. HT22). The neuroprotection has been tested following several insults (i.e. induced by glutamate, HO₂, LPS-TNFα and cytotoxic Aβ peptides), which are known to induce oxidative stress and consequently apoptosis and cell death. MT66 was also investigated as promoter of autophagy (ATG), a complex and finely regulated mechanism, essential for the correct cellular physiology. ATG is involved in the elimination of misfolded proteins, protein complexes, or damaged organelles through lysosomial degradation. Since impairments of the ATG process are associated with several neurodegenerative disorders, the ability of MT66 to promote autophagy in U-87MG cell line (an immortalized glioblastoma derived glial cells characterized by a weak ATG machinery due to the upregulation of m-TOR) was evaluated [17].

### Materials and Methods:

Amperometric Determination of H₂S: The characterization of the H₂S-generating properties of the new compounds was carried out by amperometric approaches through an Apollo-4000 Free Radical Analyzer (WPI) detector and H₂S-selective minielectrodes. The procedure has been recently reported by us [18].

Cell proliferation/viability assays: Following incubation time, cell viability was determined using the MTS assay according to the manufacturer's instruction. The dehydrogenase activity in active mitochondria reduced the 3-(4,5-dimethylthiazol-2-yl)-5-(3-carboxymethoxyphenyl)-2-(4-sulfophenyl)-2H-tetrazolium (MTS) to the soluble formazan product. The absorbance of formazan at 490 nM was measured in a colorimetric assay with an automated plate reader. The results were calculated by subtracting the mean background from the values obtained from each test condition, and are expressed as the percentage of the control (untreated cells).

[³H]MK-801 Binding Assay. Crude synaptic membranes were prepared from the cerebral cortex of Sprague-Dawley rats [19]. The pellets were stored at −80° C. for at least 24 h, and washed three more times with Tris-HEPES buffer (Tris 4.5 mM, HEPES 5 mM, pH 7.4) to remove the endogenous amino acids before the binding assay [20]. In the binding assay, 50 μL of membrane preparation (40-50 μg protein), and 10 μL of compound were mixed at 25° C. in the presence of 50 μM L-glutamate (10 μM) and 50 μL of glycine (10 μM). Then, 50 μl of [³H]MK-801 (final concentration 3 nM) were added to the preparation. Tris-HEPES buffer was added to a final volume of 0.5 mL. Following incubation time of 2 h at 25° C., binding was terminated by filtration using. Radioactivity was measured using a PerkinElmer liquid scintillation counter. Nonspecific binding was determined in the presence of unlabeled 100 μM MK-801. The dissociation constant (Kd) of [³H]MK-801 in rat cortex membranes was 4.0 nM. For compound activity determination, aliquots of membrane pellets were incubated with different ligand concentrations of MT66 and reference drug (10 nM-10 μM) in the absence of presence of 4 mM Cysteine for 30 min, and then incubated with 3 nM [³H]MK-801 for 2 h at 25° C. Samples were then filtered, and the radioactivity was counted.

The Thioflavin T fluorescence assay. Thioflavin T (ThT) dye fluorescence was used to quantify the formation and inhibition of amyloid oligomers in the presence of anti-amyloidogenic compounds. The ThT stock solution was prepared by adding 8 mg ThT to 10 mL phosphate buffer (10 mM phosphate, 150 mM NaCl, pH 7.0) and filter through a 0.2 μm syringe filter. This stock solution should be stored in the dark and is stable for about one week. On the day of the analysis, the stock solution was diluted into PBS to obtain the concentration of 10 μM. The Aβ₁₋₄₂ oligomers were prepared by diluting the stock solution in PBS and the dilution was shaking for 48 hours at 37° C. The cells were seeded in black 96-multiwell plate (3000 c/w) and treated with the compound MT66 and reference drug (10 μM) for 24 h. Following incubation time, cells were washed and incubated with Aβ₁₋₄₂ oligomers for 24 h. After this time, 200 μl of ThT 10 μM were added to each well in the dark. The ThT fluorescence intensity of each sample was recorded every 5 min using a spectrophotometer by excitation at 355 nm and emission 535 nm.

Cell proliferation/viability assays on rat microglia cells. Rat microglia cells were isolated from mixed cell culture obtained from Sprague-Dawley rat cortex, by gentle physical shaking, as described [21]. After isolation, cells were seeded into 96 well-plated with fresh culture media and pretreated with MT66 or native drug (10 μM) for 24 h; before washing and incubation with Aβ₁₋₄₂ for 24 h. Following incubation time, cell viability was determined using the MTS assay. The absorbance of formazan was measured at 490 nM. The results were calculated by subtracting the mean background from the values obtained from each test condition and were expressed as the percentage of the control (untreated cells).

Statistical analysis. Graph-Pad Prism (GraphPad Software Inc., San Diego, Calif.) was used for data analysis and graphic presentations. All data are presented as the mean±SEM. One-way analysis of variance (ANOVA) with Bonferroni's corrected t-test for post-hoc pair-wise comparisons was used to perform statistical analysis.

Mitochondria ROS generation: The generation of ROS was assessed by the fluorogenic probe DCFH2-DA (Molecular Probes, Invitrogen) in Neuronal-like cells, differentiated from H9-derived NSCs.). DCFH2-DA is a reduced and acetylated form of fluorescein used as an indicator for ROS in cells. This nonfluorescent molecule is readily converted to a green-fluorescent form (FDA) when the acetate group is removed by intracellular esterases and an oxidation by ROS occurs within the cell. Neuronal-like cells were seeded in 96-wells plate and treated with 10 μM of the compounds for 24 h. After drug removal, cells were incubated with LPS and TNF-α for an additional 16 h. One hour prior to completion of the treatment, 50 μM DCFDA was added in the same media in the dark at 37° C.; H₂O₂ was added at 100 μM as a positive control. Fluorescence increase was estimated in a plate reader at 485 nm (excitation) and 520 nm (emission) (Wallac, Victor 2, 1420 multilabel counter, PerkinElmer). The fluorescence values were normalized between samples for cell number content and assessed by a crystal violet cell staining assay. The data are expressed as percentage versus control cells.

Western blot analysis. The human U-87MG cells were seeded in 6-well plates in a final volume of 2 ml/well at a density of 1×106/well and grown to 80% of confluence with standard medium (DMEM-High Glucose). Cells were treated with vehicle (0.1% DMSO) or test compounds (i.e. 10 μM Memantine or Memit and (1 μM) Rapamycin and incubated at 37° C. for 4 and 24 h. Treated cells were washed twice with PBS and lysed in Tris-buffered saline buffer-1% Triton-X100; NaCl 150 mM; Tris-HCl 20 mM; EDTA 1 mM; EGTA 1 mM; NaF 20 mM; Na4P2O7 25 mM; Na3VO4 1 mM; PMSF 1 mM; 8 μ/ml protein cocktail inhibitors (Sigma-Aldrich, Milan, Italy). Proteins (30-40 μg) were separated on Criterion TGX™ gel (4-20%) and transferred on Immuno-PVDF membrane (Bio-Rad, Milan, Italy) for 1 h. Blots were incubated for 12 h with diluted primary antibody [1:1000, LC3A/B; p62; mTOR, p-mTOR; Akt, p-Akt(Ser 473); β-actin, Cell Signaling] in 5% w/v BSA, 1× TBS and 0.1% Tween 20 at 4° C. under gentle shaking. Then, blots were washed three times for 10 min with 1× TBS, 0.1% Tween 20 and incubated for 1 h with secondary antibody (peroxidase-coupled anti rabbit in 1× TBS, 0.1% Tween 20). After washing three times for 10 min the reactive signals were revealed by enhanced ECL Western Blotting analysis system (Amersham). Band densitometric analysis was performed using Image Lab Software (Bio-Rad, Milan, Italy).

MTT assay: HepG2 cells were seeded in a 96-well plate (Corning, USA) at a density of 1.0×104 cells/well with DMEM (200 μl/well), and then incubated for 24 h according to routine procedure. After being treated with test compounds (1-10 μM) and incubated for 24 h (8 wells for each sample), 20 μL/well MTT (5 g/L) was added to each well. The medium was then removed after 4 h incubation and 100 μL/well sodium dodecyl sulfate (SDS)-HCl solution was added to dissolve the reduced formazan product. Finally, the plate was read at 570 nm, using a micro-plate reader (Bio-Rad 680, USA).

Results

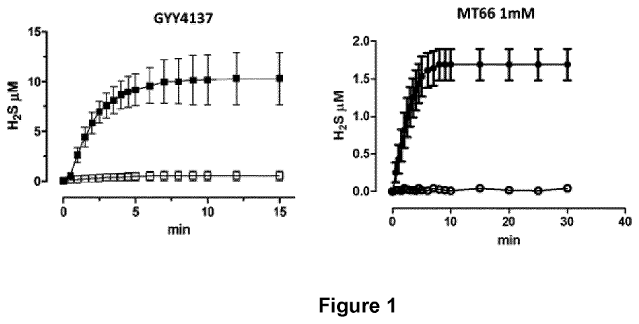

The H₂S-releasing properties were evaluated both in the absence or in the presence of an organic thiol such as L-cysteine. The new compound MT66 showed a slow and L-cysteine-dependent H₂S-releasing mechanism, similar to that exhibited by the reference slow H₂S-releasing agents, such as the phosphinodithioate derivative GYY4137 (FIG. 1). The amperometric analysis of the H2S-donor properties showed a prolonged and persisting release.

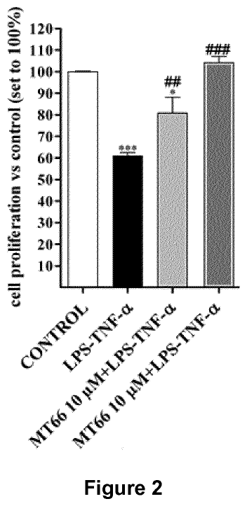

Evaluation of neuroprotective effects. The compound MT66 exhibited neuroprotective effects during inflammation. H9-derived human neural stem cells (NSCs) were differentiated to neuronal-like cells and then challenged with lipopolysaccharide (LPS) and TNF-α to establish a human in vitro model of neuroinflammation, as previously reported [19]. The effects of H₂S prodrug under physiological conditions and under inflammatory stress exposure were assessed by measuring cellular viability. Data are reported in FIG. 2. Pre-treatment for 24 or 72 h of neuronal-like cells with the selected compounds significantly counteracted the decrease in cell proliferation elicited by the inflammatory insult. The results suggest that the new compounds exert neuro-protective effects in an experimental model of inflammation.

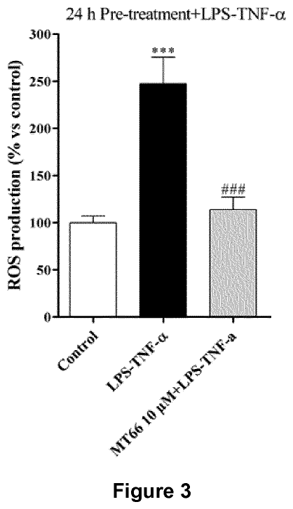

Evaluation of antioxidant activity. Aimed at investigate also the intracellular efficiency of H2S-prodrug as protective agents against ROS damage, measurement of ROS level in neuronal-like cells under inflammatory conditions was performed. FIG. 3 shows that challenging neuronal-like cells with LPS-TNF-α significantly enhanced ROS accumulation. Pre-treatment with compound MT-66 for 24 h almost completely counteracted inflammatory-mediated effects, thus suggesting that these compounds are able to prevent ROS accumulation.

Displacement of specific [³H]MK-801 binding in rat cortex membranes in the absence or presence of cysteine (Cys). MT66 was tested to assess its abilities to inhibit native-target activity (i.e. NMDAR). Binding to specific receptors (such as NMDAR) has been assessed using the assay based on the displacement of [³H]MK-801 [19]. The evaluation has been performed in the presence and in absence of Cys. The results indicated that MT66 is a prodrug of memantine (Table 1).

TABLE 1

| Displacement of specific [3H]MK-801 binding in rat | | |
|---|---|---|
| cortex membranes in the absence or | | |
| presence of cysteine (Cys). Data are reported as the means ± | | |
| S.E.M. of three different experiments (performed in duplicate). | | |
| Compound | Ki, nM (−Cys)^{a} | Ki, nM (+Cys 4 mM, 30 min)^{a} |
| MT-66 | 28.0% ^{b} | 458.4 ± 77.7 |
| memantine | 954.8 ± 74.2 | 328.8 ± 10.9 |

^{a}The Ki values are means ± SEM derived from an iterative curve-fitting procedure (Prism program, GraphPad, San Diego, CA).
^{b} Percentage of inhibition is reported for MT-66 in the absence of Cys.

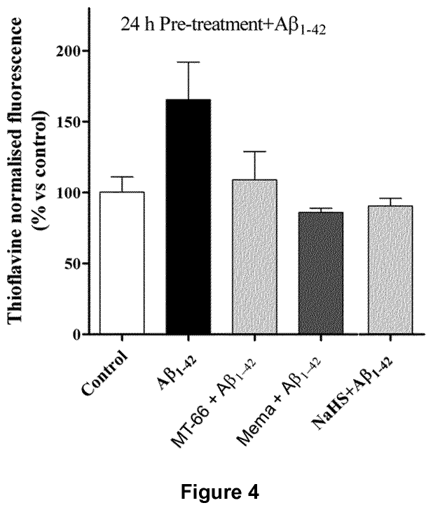

Inhibition of Aβ(1-40) self-induced aggregation. The new compound was also tested to evaluate their ability to inhibit the Aβ(1-40) self-induced aggregation. FIG. 4 shows that pre-treatment with MT-66 and the reference drugs induces a reduction of the Aδ(1-40) self-induced aggregation.

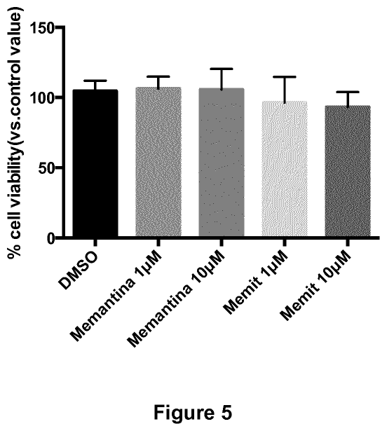

Cell viability assay: A toxicity test based on 3-(4,5-dimethylthiazol-2-yl)-2,5-diphenyltetrazolium bromide (MTT) gave information on the capability of test compounds to protect the cells against oxidative stress, in terms of viability or proliferation. The assay was performed on HepG2 cells. The cells were incubated for 24 h. Results represent cell viability and are given as percentage relative to untreated controls (FIG. 5). Any significant cytotoxicity was observed at 1 and 10 82 M .

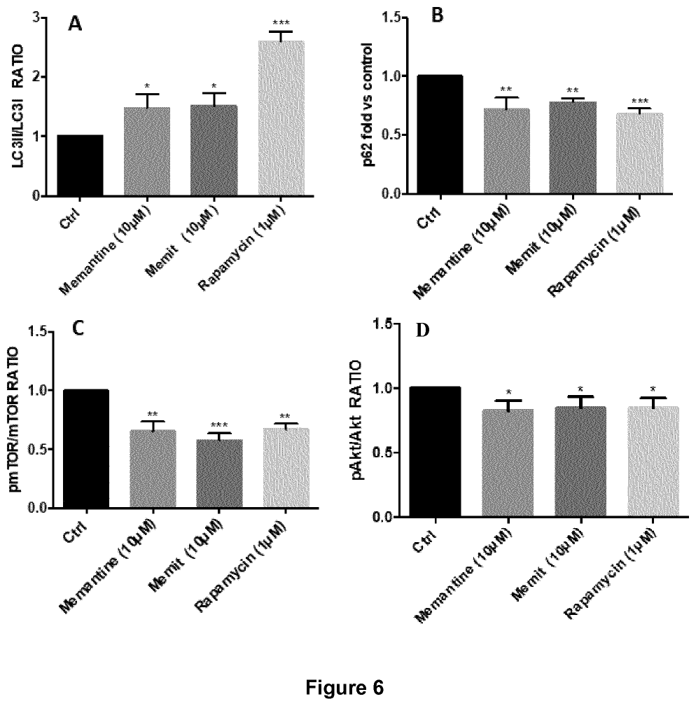

Evaluation of proautophagic activity: Expression of protein LC3-II, p62, and m-TOR, indicators of autophagy, were detected by western blotting, using Rapamycin as a positive control. In U87MG cell lines. A significant up-regulation of LC3II expression was observed after 4 h treatment with 10 μM MT66 or Memantine, and also remained almost unchanged until 24 h treatment, suggesting that both Memit and Memantine are able to stimulate the ATG flux (FIG. 6). Parallel decreased expressions of p62, which is degraded during autophagy, and p-mTOR were also observed (FIG. 6B-C). These data suggest that, similarly to Rapamycin, MT66 may induce autophagy through the inhibition of the mTOR phosphorylation by the PI3K/AKT/mTOR pathway.

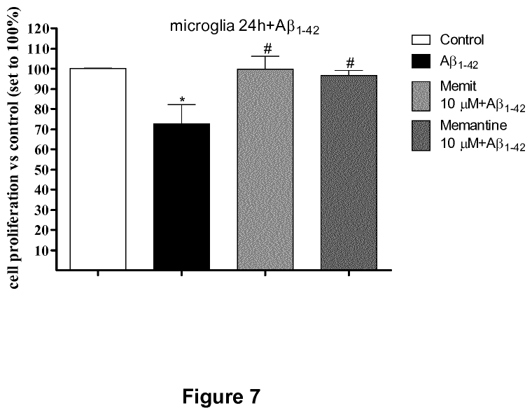

Microglia protection from Aβ(1-42) induced injury. Microglia, together with astrocytes, forms the main active immune defense of the CNS. During the inflammation process, induction of NF-KB occurs, accompanied by a release of inflammatory mediators such as TNF-α, IL-6 and nitrite ions. Notably, also levels of CBS and H₂S result down-regulated [22]. Consistently, to further determine the neuroprotection elicited by MT66, we decided to evaluate effects induced on rat microglia cells. Cells were pretreated with the compounds (10 μM concentration) followed by incubating Aβ1-42 oligomers. Pre-treatment with MT66 and Memantine completely restore cell proliferation (FIG. 7).

From the above description and the above-noted examples, the advantages attained by the compounds described and obtained according to the present invention are apparent. The present invention therefore resolves the above-lamented problem of restoring H₂S levels in the CNS, delaying the neurodegeneration process linked to inflammation and oxidative stress processes and consequently, neurodegenerative disease progression. The compounds described herein offer at the same time numerous other advantages, including making possible the development of pharmaceutical products which may be used in combination with drugs already used in the treatment of cognitive decline, allowing an improved neuroprotective pharmacological profile.

### REFERENCES

1. Wang, R., Hydrogen sulfide: the third gasotransmitter in biology and medicine. Antioxidants & redox signaling, 2010. 12(9): p. 1061-1064.

2. Zhang, X. and J.-S. Bian, Hydrogen sulfide: A neuromodulator and neuroprotectant in the central nervous system. ACS chemical neuroscience, 2014. 5(10): p. 876-883.

3. Kida, K., et al., Inhaled hydrogen sulfide prevents neurodegeneration and movement disorder in a mouse model of Parkinson's disease. Antioxid Redox Signal, 2011. 15(2): p. 343-52.

4. Kimura, H., Hydrogen sulfide as a neuromodulator. Molecular neurobiology, 2002. 26(1): p. 13-19.

5. Eto, K., et al., Hydrogen sulfide is produced in response to neuronal excitation. The Journal of neuroscience, 2002. 22(9): p. 3386-3391.

6. Kimura, H., Physiological role of hydrogen sulfide and polysulfide in the central nervous system. Neurochemistry international, 2013. 63(5): p. 492-497.

7. Nagai, Y., et al., Hydrogen sulfide induces calcium waves in astrocytes. The FASEB journal, 2004. 18(3): p. 557-559.

8. Kimura, Y., Y.-I. Goto, and H. Kimura, Hydrogen sulfide increases glutathione production and suppresses oxidative stress in mitochondria. Antioxidants & redox signaling, 2010. 12(1): p. 1-13.

9. Jia, J., et al., Differential mechanisms underlying neuroprotection of hydrogen sulfide donors against oxidative stress. Neurochemistry international, 2013. 62(8): p. 1072-1078.

10. Xie, Z.-Z., et al., Sulfhydration of p66Shc at cysteine59 mediates the antioxidant effect of hydrogen sulfide. Antioxidants & redox signaling, 2014. 21(18): p. 2531-2542.

11. Fan, H., et al., Hydrogen sulfide protects against amyloid beta-peptide induced neuronal injury via attenuating inflammatory responses in a rat model. Journal of Biomedical Research, 2013. 27(4): p. 296-304.

12. Moore, P. K., M. Bhatia, and S. Moochhala, Hydrogen sulfide: from the smell of the past to the mediator of the future? Trends in pharmacological sciences, 2003. 24(12): p. 609-611.

13. Eto, K., et al., Brain hydrogen sulfide is severely decreased in Alzheimer's disease. Biochemical and biophysical research communications, 2002. 293(5): p. 1485-1488.

14. Liu, Y. and J. Bian, Hydrogen sulfide protects amyloid-β induced cell toxicity in microglia. Journal of Alzheimer's disease: JAD, 2010. 22(4): p. 1189.

15. Barresi, E., et al., Iminothioethers as Hydrogen Sulfide Donors: From the Gasotransmitter Release to the Vascular Effects. Journal of Medicinal Chemistry, 2017.

16. Rapposelli, S., et al., A Novel H2S-releasing Amino-Bisphosphonate which combines bone anti-catabolic and anabolic functions. Sci Rep, 2017. 7(1): p. 11940.

17. Catalano, M.; et al. Autophagy induction impairs migration and invasion by reversing EMT in glioblastoma cells. Mol Oncol 2015, 9 (8), 1612-25.

18. Martelli, A., et al., Arylthioamides as H2S donors: L-cysteine-activated releasing properties and vascular effects in vitro and in vivo. ACS Medicinal Chemistry Letters, 2013. 4(10): p. 904-908.

19. Daniele, S., et al., Trazodone treatment protects neuronal-like cells from inflammatory insult by inhibiting NF-kB, p38 and JNK. Cellular signalling, 2015. 27(8): p. 1609-1629.

20. Simoni, E., et al., Combining Galantamine and Memantine in Multitargeted, New Chemical Entities Potentially Useful in Alzheimer's Disease. Journal of Medicinal Chemistry, 2012. 55(22): p. 9708-9721.

21 Daniele, S., et al. Human Neural Stem Cell Aging Is Counteracted by α-Glycerylphosphorylethanolamine ACS chemical neuroscience 2016, 7, p. 952-963

22. Wojtera, M., et al. Microglial cells in neurodegenerative disorders. Folia neuropathologica 2005, 43, p. 311-321

1. A compound of formula (I) 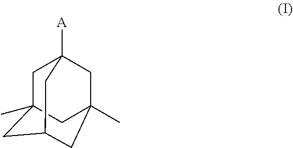
or a pharmaceutical salt thereof
wherein:
A is —N═C═S or NH—B,
where B is 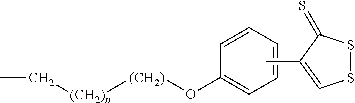
and n=0-3
for use in the treatment of a neurodegenerative disease.

2. The compound for use according to claim 1, wherein A is —N═C═S.

3. The compound for use according to claim 1, wherein A is —NH—B.

4. The compound for use according to claim 3, wherein n is preferably 1.

5. The compound for use according to claim 1, wherein the neurodegenerative disease is a disease selected from the group consisting of Parkinson's disease, Alzheimer's disease amyotrophic lateral sclerosis and Huntington's disease

6. A compound of formula (I) 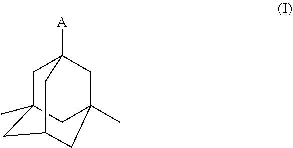
or a pharmaceutical salt thereof
wherein:
A is —NH—B,
where B is 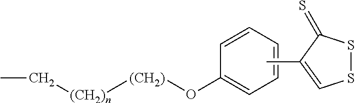
and n=0-3.

7. The compound of claim 6, wherein n is 1.

8. A pharmaceutical composition comprising 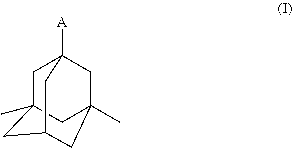
or a pharmaceutical salt thereof
wherein:
A is —N═C═S or —NH—B
where B is 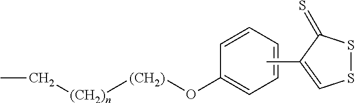
and n=0-3
and Memantine of Formula

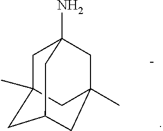

9. The pharmaceutical composition according to claim 8, wherein A is —N═C═S.
